(19) United States
(12) Patent Application Publication
(10) Pub. No.: US 2022/0001202 A1
(43) Pub. Date: Jan. 6, 2022

(54) APPARATUS FOR PRODUCING A STERILIZED ANIMAL POPULATION

(71) Applicant: ZYZZLE LTD, Surrey (GB)

(72) Inventor: Glen SLADE, Surrey (GB)

(21) Appl. No.: 17/291,310

(22) PCT Filed: Nov. 22, 2019
(86) PCT No.: PCT/GB2019/053308

§ 371 Date: May 5, 2021

(30) Foreign Application Priority Data
Nov. 26, 2018 (GB) 1819190.8

(51) Int. Cl. A61N 5/10; A01M 99/00; A61D 99/00

(52) U.S. Cl. CPC A61N 5/10; A61N 2005/1094; A61D 99/00; A01M 99/00

(57) ABSTRACT

Apparatus for shielding a non-human animal from a sterilizant. The apparatus (100, 200) defines a space (102, 202) for accommodating a non-human animal (111, 204) and a shield (107, 201) for shielding a part of the animal from a sterilizant such as radiation. Only exposing a specific part of the animal to sterilizant produces a sterile animal which is better adapted to mate with other animals When the sterilized animals are released in a sterile insect technique, the technique is more efficient because the released animals are better adapted to mate with a wild population.

The present invention provides apparatus for producing an improved animal population, in particular an improved population of sterilized animals. A method for producing an improved animal population is also provided.

Many animals, particularly insects, need to have their wild populations controlled for the benefit of human welfare or other objectives. Examples are public health nuisances, such as mosquitoes that transmit diseases, and agricultural insect pests that cause crop losses.

One possible method of controlling animal populations involves rearing and releasing a large number of sterile males of that species. Once these sterilized male animals are released into the wild, they compete with wild male animals to mate. Since mating events involving the sterile animals will not produce offspring, the population of the wild female animals is reduced over time. This can deliver benefits associated with the planned population reduction. In the context of insects, this technique is known as the ‘sterile insect technique’ (SIT). Similar methods can also be used to control other target animal populations, whether or not those animals spread diseases or damage crops.

Conventionally when insects are reared for the SIT, for example the Mediterranean fruit fly (Ceratitis capitata), they are sterilized at the pupa stage of development using ionizing radiation. In variations on the SIT, the sterilization may be achieved by chemosterilizants and/or applied to a different life stage of the animal.

Typical current industrial scale processes sterilize batches of thousands of pupae which are contained in a container having a volume of roughly one litre. The sterilization may be achieved by subjecting pupae in the volume to ionizing radiation. A drawback of this approach is that the ionizing radiation has a detrimental effect on the insect, both reducing the percentage of pupae that become adults and the fitness of the sterilized adults in terms of ability to successfully find and mate with wild insects. This reduces the effectiveness of the technique and/or raises the costs of the technique, because more insects need to be reared and released to have the desired effect.

These problems exist generally for variations on the SIT including other sterilization methods and other similar techniques for controlling the population of other types of animals.

It is amongst the objects of the invention to address one or more of these problems.

In a first aspect the invention provides an apparatus for shielding a non-human animal from a sterilizant comprising;
a space for accommodating a non-human animal, and
a shield for shielding part of a non-human animal which is accommodated in the space from a sterilizant.

In many situations, only the genitalia of the animal need to be exposed to a sterilization treatment for the animal to be sterilized. The presence of the shield allows parts of the animal other than the genitalia to be shielded from the treatment which is used to sterilize the animal. For example a shield could protect an animal's body from ionizing radiation. This avoids the deleterious effect of the radiation on the other body parts of the animal. When the animal is fully developed, this will provide an adult animal which is better adapted to mate with a wild population than animals which have been subject to blanket radiation. The improved characteristics of a sterile animal population produced using the apparatus means that fewer animals need to be reared to have the same effect when released into the wild. This can reduce the costs associated with the process and/or provide a more efficient reduction of the wild population which needs to be controlled.

In the following, some embodiments described use (or are apparatus for use with) sterilization by irradiation. This does not imply any limitation of the invention with respect to other methods of sterilization. Other methods of sterilization may be used and the apparatus described herein may be suitable for use with other methods of sterilization.

The shield may be a radiation shield. In the context where sterilization is effected by irradiation, preferably the radiation shield comprises a material which is substantially impermeable to ionizing radiation such as lead. Preferably the radiation shield substantially blocks all radiation (which is incident upon it) from passing through it. To obtain the most desirable results the percentage of radiation which needs to be blocked will depend on the species of animal with which the apparatus or method will be used. The preferred percentages of radiation blocked may also depend upon the type of radiation used. In some embodiments 20% to 95%, and preferably 50% to 98%, of incident radiation from a particular radiation source is blocked by the radiation shield.

The apparatus may comprise a plurality of spaces for accommodating non-human animals separately. This allows for an individualized, predictable and controllable dose of radiation to be applied to each animal. It also avoids any effects associated with one animal blocking or refracting radiation which would otherwise be incident upon another animal in a given container. The spaces may be an array of containers. The containers may be formed as a microtitre plate.

The volume of the space, or each space, may be no greater than 0.001 m³. The volume of the space, or each space, may be less than 0.0001 m³ or less than 0.00001 m³. The volume of the space, or each space, may be within the range of 1.5×10⁻⁸ m³ to 3.6×10⁻⁷ m³. In some embodiments the sum of the sides (height+width+breadth) of the space, or each space, is no greater than 30 cm, or no greater than 20 cm, or no greater than 10 cm. The ratio of the width to the length to the depth of the space defined by each well may be about 1:1:1.5

The apparatus may further comprise a means for moving the space or the plurality of spaces through radiation or a chemosterilizant. This allows the apparatus to operate as a continuous process in which a radiation source or chemosterilizant source does not need to be switched on and off for discrete batches of animals.

The means for moving the space or the plurality of spaces through radiation or chemosterilizant may be a conveyor. The means for moving the space or the plurality of spaces may follow a curved path. The path may be a linear or non-linear path which extends over 2 or 3 space dimensions. For example the path may be generally circular (i.e. over 2 dimensions) or the path may be a generally spiral path (i.e. over 3 dimensions). This allows animals to orbit a sterilizant source when the sterilizant source emits sterilizant over a disperse volume rather than, for example, via a narrowly directed beam. The space or spaces may be formed as containers and may be shaped to hold animals in a ring-type arrangement around a sterilizant source.

The shield is preferably a radiation shield which is positioned as close to the animal as is practical to reduce refraction of incident radiation. Preferably, when an animal is accommodated in the space, the average distance between the animal and a wall which defines the space is less than 1 mm, less than 2 mm, less than 5 mm or less than 10 mm.

The space may be elongate so that the movement of a non-human animal accommodated in the space is restricted. Many insect pupae have an elongate shape. In some embodiments the elongate shape of the space is adapted to prevent a pupa from being inverted once it is accommodated in the space. This restriction of movement assists in exposing part of the animal to a predictable dose of sterilizant, which is incident substantially only upon the parts of the animal which cause sterilization. For insect pupae, the genetalia are normally located near to an end of the elongate shape of the pupa. This allows the genetalia to protrude clearly from the part of the space which is shielded by the shield.

The apparatus may further comprise a device for lowering the temperature within the space.

In some embodiments the animal is exposed to sterilizant, for example ionizing radiation, for many seconds or a few minutes. It is preferable that one can be confident that the animal will not move substantially over this timeframe. This is particularly important because it can be difficult to put cameras inside the equipment to check whether the animal has moved. Chilling the space in which the animal is accommodated may be used to immobilize the animal. This is particularly useful for immobilising insects.

The space may allow for degree(s) of freedom of movement of the animal, provided that this freedom of movement does not compromise the consistent exposure of the desired parts of the animal to sterilizant.

Other aspects of the space or actions taken on the animal may be adapted to immobilise the animal. These actions may include treatments applied to the animal, for example the administration of a sedative.

The space may be formed in the shield. This allows a one piece apparatus. In some embodiments this will facilitate manufacture and reduce the costs of production as the spaces may be formed, for example as recesses by drilling, in a single block of material. The space may be formed as a well. This allows an animal to be deposited in the space easily without removing a covering. It also allows the introduction of fluid or other material into the space.

The space may be formed as a well and the shield may form a removable lid which is adapted to be fitted over the well. This enables easy filling of the space with an animal. The lid configuration may also restrict a mobile animal from moving during the process. Providing a lid in embodiments which comprise many spaces for accommodating animals, enables the application of a shield to many spaces at the same time. This saves time and provides a more efficient method. Providing the shield in the form of a lid may also reduce the costs of manufacture of the apparatus. This contrasts with embodiments in which the spaces are formed in a block of shield material. Generally, less expensive shielding material is required for embodiments in which the shield is configured as a lid, rather than as a block in which the spaces are provided.

The space may contain a fluid. The fluid may float an animal in the space, for example a pupa, so that the position of the animal with respect to a sterilizant source is optimized. The position of the animal may be altered/optimized by adding or removing fluid from the space. Equally, fluid may be necessary for the survival of the animal. Chilled fluid, such as water, may also be used to reduce mobility of the animal.

The shield may be a radiation shield and the apparatus may further comprise a source of ionizing radiation. The source of ionizing radiation may produce X-Rays, gamma rays, other electromagnetic radiation or alpha particle radiation. The radiation provides a sterilization effect when incident upon certain parts of animals. The source of ionizing radiation and the radiation shield may be arranged such that only a selected part of a non-human animal accommodated in the space is exposed to radiation. In preferred embodiments only the genitalia of the animal will be exposed to the ionizing radiation. The size, shape and configuration of the radiation shield will vary according to the animal used.

The apparatus may comprise a source of chemosterilizant. The source of chemosterilizant and the shield may be arranged such that only a selected part of a non-human animal accommodated in the space is exposed to chemosterilizant. The chemosterilizant may be applied to the non-human animal by immersing or partially immersing a part of the non-human animal in the chemosterilizant.

In some embodiments, the shield is provided with an aperture through which radiation or chemosterilizant may pass to become incident upon an animal accommodated in the space.

In a second aspect the invention provides an apparatus for exposing part of a non-human animal to a sterilizant comprising;
a body which defines a space for accommodating a non-human animal,
wherein the body is adapted to support a non-human animal present in the space in an orientation which permits a part the non-human animal to be introduced into a bath of liquid sterilizant, without other parts of the non-human animal being exposed to the liquid sterilizant.

The body may be formed by a mesh support. This allows for liquid sterilizant to easily flow into the space and come into contact with part of a non-human animal accommodated within the space. The body may comprise an array of wells, each of which is provided with an aperture to allow sterilizant to flow into the space and come into contact with at least part of a non-human animal accommodated in the space. Preferably the aperture is located towards a lower end of each well. The apparatus may comprise a space or spaces having the characteristics of the space or spaces described in connection with the other aspects of the invention.

In a third aspect of the invention there is provided a combination of an apparatus according to the second aspect of the invention and a bath for holding liquid sterilizant.

In the combination, when a non-human animal is present in the space and the apparatus is deposited in the bath, the bath may be filled with sterilizant and the depth of the bath may be arranged so that even when the bath is full of sterilizant, a portion of the non-human animal is held above the sterilizant level. This provides a failsafe mechanism to ensure that parts of the non-human animal other than its genitalia cannot exposed to the sterilizant by overfilling the bath.

The apparatus may be permanently attached to the bath. The apparatus may comprise a well, the lower end of which is open-ended so that a non-human animal which is deposited in the well falls into sterilizant which is present in the bath. The walls of the well may still however hold the non-human animal in a pre-defined orientation, such as generally upright, when the non-human animal drops into the sterilizant. This means that only the genitalia of the animal are exposed to the sterilizant.

In a fourth aspect the invention provides a method of preparing a non-human animal for sterilization comprising the step of depositing a non-human animal into the space of an apparatus or a combination of an apparatus and a bath as described herein.

The method may comprise a further step of exposing a part of the non-human animal to radiation or a chemosterilizant.

The non-human animal may be an arthropod. The non-human animal may be an insect. The non-human animal may be at any life stage including the stages of egg, larva, pupa or adult. The animal may be a rodent, such as a rat.

The method may be carried out in a low temperature environment, for example at a temperature below 10° C.

In a fifth aspect the invention provides a sterile animal technique comprising the steps of subjecting a non-human animal to a method of sterilization described herein, and releasing the non-human animal into the wild. Preferably the non-human animal is an insect.

Features of the apparatus and combination of the apparatus and a bath described herein may be incorporated in methods described herein. The methods described herein may contain the steps of using any of the features of the apparatus or the combination of the apparatus and bath described herein. Equally, the apparatus or combination of the apparatus and bath according to the invention may incorporate features which are adapted to perform the features of the methods described herein. Features from different aspects of the invention may also be combined.

Non-limiting embodiments of the invention will now be described with reference to the drawings, in which:

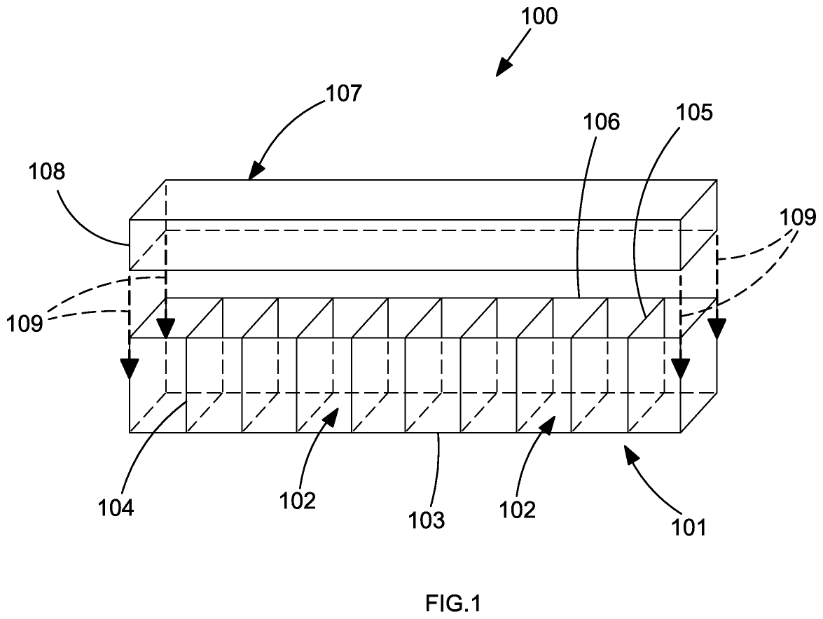

FIG. 1 shows a perspective transparent view of first embodiment of the invention.

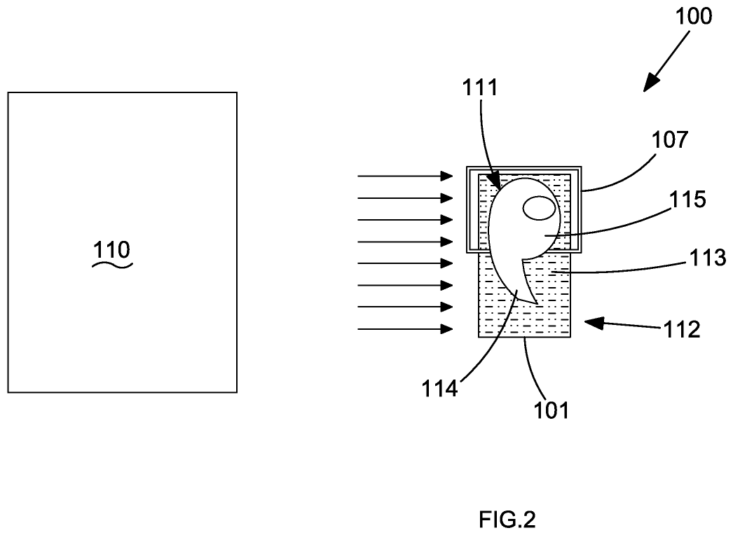

FIG. 2 shows a side sectional view of a pupa accommodated in the apparatus shown in FIG. 1, in combination with a radiation source.

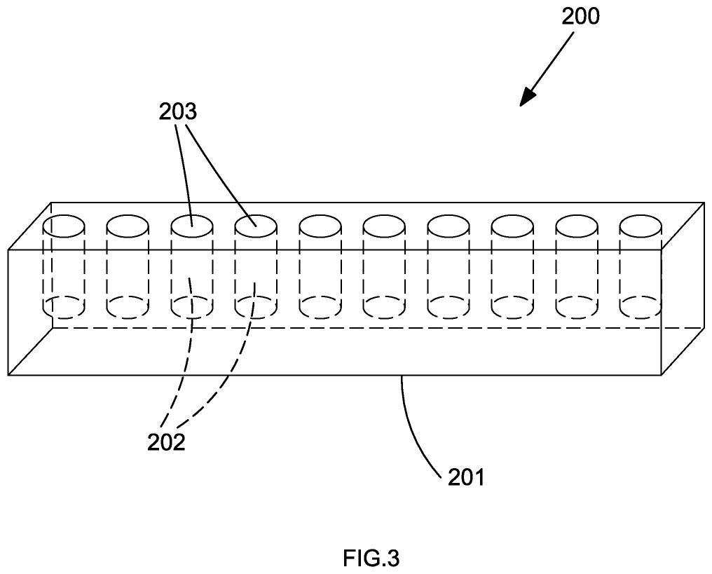

FIG. 3 shows a perspective transparent view of a second embodiment of he invention.

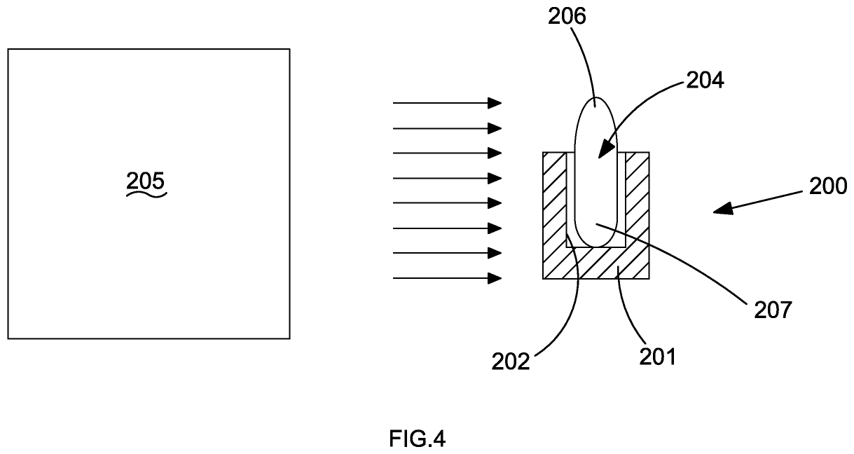

FIG. 4 shows a sectional view of a pupa accommodated in the apparatus shown in FIG. 3, in combination with a radiation source.

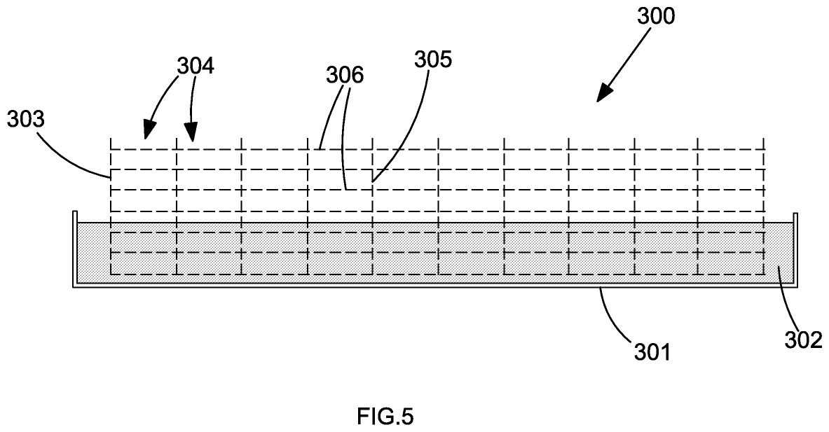

FIG. 5 shows a cross sectional front view of a first embodiment of a combination of an apparatus and a bath according to the invention.

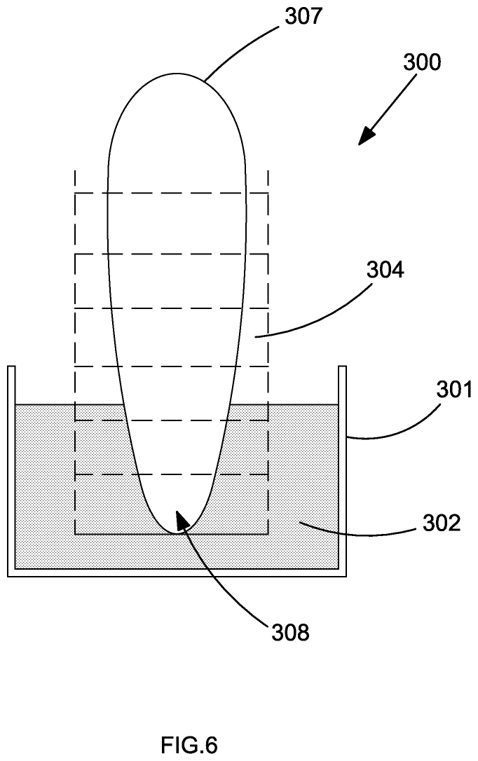

FIG. 6 shows a cross sectional side view of the embodiment shown in FIG. 5.

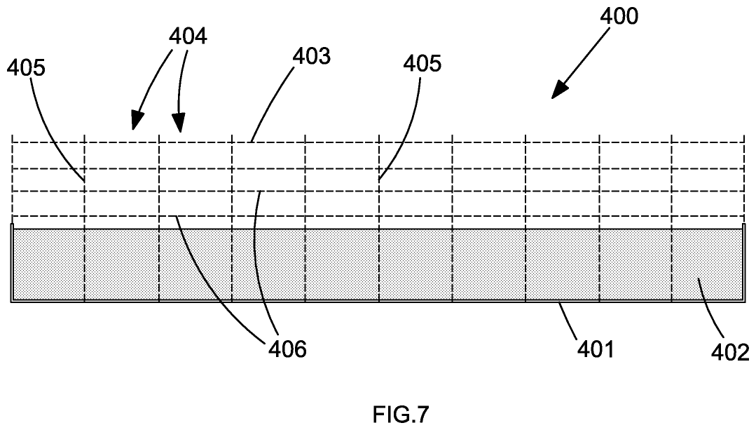

FIG. 7 shows a cross sectional front view of a second embodiment of a combination of an apparatus and bath according to the invention.

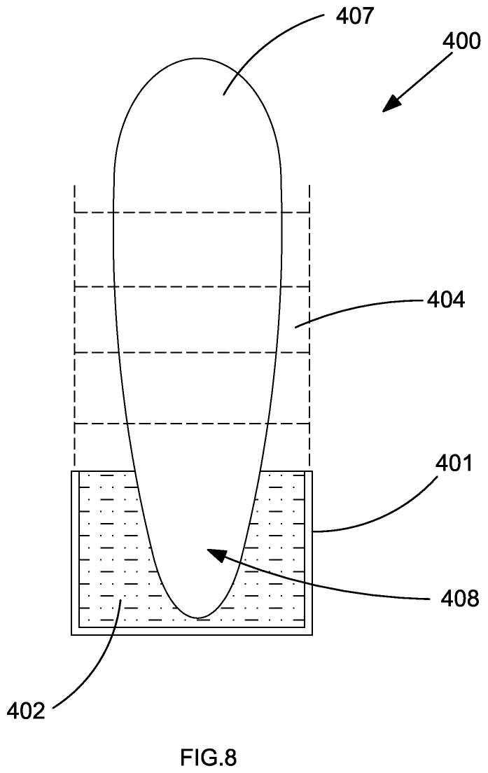

FIG. 8 shows a cross sectional side view of the embodiment shown in FIG. 7.

FIG. 1 shows an apparatus 100. The apparatus comprises a tray 101 which is provided with ten wells 102. The volume of each of the wells has a rectangular cross section when viewed from above. The wells form a linear array. Each well has a flat bottom 103 and an open mouth at its upper end. Each well has a depth 104 that is larger than its width 105 and larger than its length 106. The ratio of width to length to depth of the volume defined by each well is about 1:1:1.5. The wells are arranged side by side so that each of the wells is adjacent to two other wells, except for the end wells, which are only adjacent to one other well. The tray is made of a plastic material which is substantially transparent to ionizing radiation.

A radiation shield 107 is also provided. The radiation shield has the shape of a hollow rectangular cuboid which is missing its lower face. The dimensions of the radiation shield are such that it may be removably fitted over the tray 101, along the path shown by the arrows 109. The depth 108 of the radiation shield is such that when the radiation shield is fitted over the tray 101, a lower portion of each well 102 protrudes from below the radiation shield. This is more clearly shown in FIG. 2. The radiation shield is made from lead, which is substantially impermeable to ionizing radiation.

FIG. 2 shows a side sectional view of the apparatus 100 shown in FIG. 1, in combination with a radiation source 110. A pupa 111 is accommodated in a well of the apparatus. Further pupae (not shown) are accommodated individually in each of the other wells of the tray. The radiation shield 107 is fitted over the tray 101. A lower portion of the tray 112 protrudes from the lower end of the radiation shield.

The well contains water 113, in which the pupa floats. In the apparatus shown, the width and length of the well are not chosen or arranged so that the elongate pupa is prevented from being inverted whilst in the well. In other embodiments the width and length of the well are chosen and arranged so that the elongate pupa is prevented from being inverted whilst in the well. The portion 114 of the pupa which protrudes from under the radiation shield contains the animal's genitalia. The portion 115 of the pupa which does not protrude from the radiation shield contains body parts of the animal other than its genitalia.

The radiation source 110 is arranged so that when it is switched on, gamma or X-Ray radiation, which is represented by the arrows in FIG. 2, is emitted from the radiation source and is incident upon the apparatus 100. The radiation shield is arranged such that it shields the portion of the pupa 115 from the incident radiation. The lower portion of the pupa 114 is however fully exposed to the radiation, thereby sterilizing the animal.

The radiation is applied to the apparatus and pupae for approximately one minute. The radiation source is then switched off and the pupae are removed from the wells and allowed to develop fully before they are released into the wild.

FIG. 3 shows an apparatus 200. The apparatus comprises a rectangular cuboid block 201 made of lead. The block is provided with a linear array of ten wells 202, which are formed as recesses in the top face of the block. The wells define volumes which have a circular cross section when viewed from above. At their upper ends the wells have circular mouths 203. The wells have a constant cross section over their height and are flat bottomed. The wells have heights which are larger than the diameters of their mouths.

FIG. 4 shows a side sectional view of a pupa 204 accommodated in the apparatus 200 shown in FIG. 3, in combination with a radiation source 205. Other pupae (not shown) are accommodated individually in each of the other wells of the block 201. The well 202 is sized so that the pupa is prevented from being inverted. That is, the sides of the well 202 restrict the pupa's movement so that it is held in a generally upright orientation. The depth of the well 202 is arranged so that a portion 206 of the pupa protrudes through the mouth 203 of the well 202. A portion 207 of the pupa does not protrude from the well. The portion 206 of the pupa which protrudes from the well contains the genitalia of the animal. Substantially no fluid is present in the well 202.

The radiation source 205 is arranged so that when it is switched on, gamma or X-Ray radiation, which is represented by the arrows in FIG. 4, is emitted from the radiation source and is incident upon the apparatus 200. The portion 206 of the pupa which protrudes from the well 202 is fully exposed to the radiation, whereas the portion 207 of the pupa which is contained within the well is shielded by the lead block 201 from the radiation. A similar arrangement exists with the other pupae, which are not shown in this view.

The radiation is applied to the apparatus and pupae for approximately one minute. The radiation source is then switched off and the pupae are removed from the wells and allowed to develop fully before they are released into the wild.

FIG. 5 shows a combination of an apparatus 300 which is disposed in a bath 301 which is filled with liquid sterilizant 302. The apparatus is formed from a coarse wire mesh 303. The mesh is shaped to define an array of wells 304 with square cross sections. There are ten wells which are linearly arranged so that all wells share walls with adjacent wells. In other embodiments, the wells are arranged in a 2 dimensional array. Each well is sized to receive a pupa. The wells are defined by vertical mesh struts 305 at each corner of the well and by horizontal struts 306 which bridge the vertical struts. The wells are elongated so that they can each accommodate an elongate pupa. The lower ends of each well are provided with a stop (not shown) to prevent a pupa from falling through the bottom of the well. The stop may be formed from the mesh which defines the well.

FIG. 6 shows a pupa 307 deposited in a well 304 of the apparatus 300. In use, a pupa 307 is deposited into each well 304 so that its genitalia 308 are close to the lower end of the well. This may be done when the apparatus is not located in the bath. The apparatus 300 is then lowered into the bath 301, which contains liquid sterilizant 302. A suitable liquid sterilizant is thiotepa. The level of liquid sterilizant in the bath is set so that when the apparatus 300 is either lowered into the bath by a predetermined amount, or rests upon the bottom of the bath, the pupae which are disposed in the wells are only partially immersed into the sterilizant. The level of sterilizant is set so that generally, only the genitalia of the pupae are immersed in the sterilizant. The wells are arranged so that the rest of the pupa in each well is situated above the level of sterilizant. This means that the sterilizant has less of an effect on the characteristics of the pupae (other than their reproductive ability), than would be the case if the pupae were fully submerged. This leads to an improved insect population once the pupae have developed.

FIG. 7 shows a second combination of an apparatus 400 and bath 401 according to the invention. The apparatus is formed from a coarse wire mesh 403. The mesh is shaped to define an array of wells 404 with square cross sections. There are ten wells which are linearly arranged so that all wells share walls with adjacent wells. In other embodiments, the wells are arranged in a 2 dimensional array. Each well is sized to receive a pupa. The wells are defined by vertical mesh struts 405 at each corner of the well and by horizontal struts 406 which bridge the vertical struts. The wells are elongated so that they can each accommodate an elongate pupa. The walls of the wells which are formed by the 2.0 vertical struts 405 extend into the bath 401, which is filled with liquid sterilizant 402. In other embodiments, the wire mesh does not extend into the bath and starts where the wall of the bath ends. The wells in the apparatus 400 do not have a separate stop at their lower ends. The lower ends of the wells terminate with the bottom of the bath 401.

FIG. 8 shows a pupa 407 deposited in a well 404 of the apparatus 400. In use, a pupa 407 is deposited into each well 404 so that its genitalia 408 are close to the lower end of the well. The apparatus 400 is attached to the bath 401 so that, in use, pupae may be deposited into the top end of wells 404 and fall down the wells into the sterilizant 402 which is present in the bath. The bath is filled to the brim with sterilizant. The depth of the bath is chosen so that generally only the genitalia of pupae which are accommodated in the wells are submerged in the bath. The sterilizant level is in no danger of being raised to harm other parts of pupae in the wells because any further sterilizant which is added to the bath will simply overflow the sides of the bath and the sterilizant level will not rise.

The wells are arranged so that the rest of the pupa in each well is situated above the level of sterilizant. This means that the sterilizant has less of an effect on the characteristics of the pupae (other than their reproductive ability), than would be the case if the pupae were fully submerged. This leads to an improved insect population once the pupae have developed.

1-28. (canceled)

29. Apparatus for shielding a non-human animal from a sterilizant comprising;
a space for accommodating a non-human animal, and
a shield for shielding part of a non-human animal which is accommodated in the space from a sterilizant.

30. An apparatus according to claim 29 wherein the shield is a radiation shield.

31. An apparatus according to claim 29 which comprises a plurality of spaces for accommodating non-human animals separately.

32. An apparatus according to claim 29 wherein the volume of the space, or each space, is no greater than 0.001 m³, is less than 0.0001 m³ or is less than 0.00001 m³.

33. An apparatus according to claim 29 further comprising a means for moving the space or the plurality of spaces through radiation or a chemosterilizant.

34. An apparatus according to claim 33 wherein the means for moving the space or the plurality of spaces through radiation or chemosterilizant is a conveyor.

35. An apparatus according to claim 33 wherein the means for moving the space or the plurality of spaces follows a curved path.

36. An apparatus according to claim 29 wherein the space is elongate so that the movement of a non-human animal accommodated in the space is restricted.

37. An apparatus according to claim 29 further comprising a device for lowering the temperature within the space.

38. An apparatus according to claim 29 wherein the space is formed in the shield.

39. An apparatus according to claim 29 wherein the space is formed as a well.

40. An apparatus according to claim 29 wherein the space is formed as a well and the shield forms a removable lid which is adapted to be fitted over the well.

41. An apparatus according to any claim 29 wherein the space contains a fluid.

42. An apparatus according to claim 29 wherein the shield is a radiation shield and the apparatus further comprises a source of ionizing radiation.

43. An apparatus according to claim 42 wherein the source of ionizing radiation and the radiation shield are arranged such that only a selected part of a non-human animal accommodated in the space is fully exposed to radiation.

44. An apparatus according to claim 29 further comprising a source of chemosterilizant.

45. An apparatus according to claim 44 wherein the source of chemosterilizant and the shield are arranged such that only a selected part of a non-human animal accommodated in the space is exposed to chemosterilizant.

46. Apparatus for exposing part of a non-human animal to a sterilizant comprising;
a body which defines a space for accommodating a non-human animal,
wherein the body is adapted to support a non-human animal present in the space in an orientation which permits a part of the non-human animal to be introduced into a bath of liquid sterilizant, without other parts of the non-human animal being exposed to the liquid sterilizant.

47. A combination of an apparatus according to claim 46 and a bath for holding liquid sterilizant.

48. A combination according to claim 47 wherein, when a non-human animal is present in the space and the apparatus is deposited in the bath, the bath may be filled with sterilizant and the depth of the bath is arranged so that even when the bath is full of sterilizant, a portion of the non-human animal is held above the sterilizant level.
